(19) United States
(12) Patent Application Publication
(10) Pub. No.: US 2024/0099808 A1
(43) Pub. Date: Mar. 28, 2024

(54) Systems, Devices, and Methods for Landmarking a Medical Device

(71) Applicant: Bard Access Systems, Inc., Salt Lake City, UT (US)

(72) Inventors: Steffan Sowards, Salt Lake City, UT (US); William Robert McLaughlin, Bountiful, UT (US)

(21) Appl. No.: 18/371,329

(22) Filed: Sep. 21, 2023

Related U.S. Application Data
(60) Provisional application No. 63/409,553, filed on Sep. 23, 2022.

(51) Int. Cl. A61B 90/00; A61B 34/00

(52) U.S. Cl. CPC A61B 90/39; A61B 34/25; A61B 2034/2061; A61B 2090/3958

(57) ABSTRACT

Disclosed are medical systems, devices, and methods that support landmarking of a medical device pathway within a patient. A landmarking device that is placed on the patient and coupled with a system module, includes magnetic elements and/or a shape sensing optical fiber to enable determining a position and shape of a landmarking pathway by logic of the system module. A medical device correspondingly includes magnetic elements and/or a shape sensing optical fiber that enable determining an actual pathway of the medical device by logic of the system module during insertion of the medical device. An image of the landmarking pathway combined with an image of the actual pathway are depicted on a display. The logic compares the actual pathway with the landmarking pathway and provides an alert accordingly.

### PRIORITY

This application claims the benefit of priority to U.S. Provisional Application No. 63/409,553, filed Sep. 23, 2022, which is incorporated by reference in its entirety into this application.

### BACKGROUND

The tracking of medical devices, such as intravascular medical devices within a patient, provides for enhanced accuracy of placement and reduced patient risk resulting from incorrect placement. One example is the placement of a central catheter with the superior vena cava. However, in the absence of predefined landmarks, navigating an internal pathway and confirming a correct placement of a vascular device remain prone to error.

Disclosed herein are systems, devices, and methods that address the forgoing.

### SUMMARY

Briefly summarized, disclosed herein is a medical system that, according to some embodiments, includes an elongate landmarking device configured for placement across an exterior of the patient, where the landmarking device includes a plurality of landmarking elements extending along a length of the landmarking device. The system further includes a system module comprising a console operably coupled with the landmarking device, the console having a number of processors and memory including a non-transitory computer-readable medium, having logic stored thereon that, when executed by the processors, performs operations. The operations include receiving landmarking signals originating from the plurality of landmarking elements and defining one or more digital landmarks of the landmarking device based on the received landmarking signals. The plurality of landmarking elements are arranged along the landmarking device to define the one or more digital landmarks, and the digital landmarks are configured to indicate to a desired insertion pathway for an elongate medical device.

In some embodiments, the operations further include defining a landmarking pathway based on the one or more digital landmarks, and in some embodiments, the operations further include storing the landmarking pathway in memory.

In some embodiments, the console is coupled with a display, and the operations further include depicting the landmarking pathway in combination with the actual pathway on the display.

In some embodiments, the operations further include comparing the landmarking pathway with the actual pathway and providing an alert in response to the comparison when the actual pathway deviates from the landmarking pathway beyond a defined deviation limit.

In some embodiments, the landmarking elements include landmarking magnetic elements, and the received landmarking signals include magnetic signals emanating from the landmarking magnetic elements.

In some embodiments, the landmarking device includes a shapable elongate member. In some embodiments, the landmarking magnetic elements are disposed along the shapable elongate member and in further embodiments, the landmarking magnetic elements are positionable along the shapable elongate member.

In some embodiments, the landmarking device includes a landmarking optical fiber extending along the shapable elongate member, where (i) the landmarking optical fiber includes multiple optical fiber cores having a number of optical fiber gratings disposed along the multiple optical fiber cores, (ii) the number of optical fiber gratings define reflected optical signals based on a shape of the shapable elongate member, (iii) the landmarking optical fiber is optically coupled with the console, and (iv) the received landmarking signals include the reflected optical signals propagated proximally along the optical fiber of the landmarking device.

In some embodiments, the system further includes an elongate medical device configured for insertion within a patient, wherein the elongate medical device is operably coupled with the console, and where the elongate medical device includes a number of device tracking elements disposed along the elongate medical device.

In some embodiments, the operations further include receiving by the system module device tracking signals originating from the device tracking elements disposed along the elongate medical device and determining an actual pathway of the elongate medical device inserted into the patient, where the actual pathway is based on the received device tracking signals.

In some embodiments, the console is coupled with a display and the operations further include depicting an image of the landmarking pathway in combination with an image of the actual pathway on the display.

In some embodiments, the operations further include comparing the landmarking pathway with the actual pathway and providing an alert in response to the comparison when the actual pathway deviates from the landmarking pathway beyond a defined deviation limit.

In some embodiments, the device tracking elements include tracking magnetic elements disposed along the elongate medical device, and the received device tracking signals include magnetic signals emanating from the tracking magnetic elements.

In some embodiments, the elongate medical device includes a device optical fiber extending along the elongate medical device, where (i) the device optical fiber includes multiple device optical fiber cores having a number of device optical fiber gratings disposed along the multiple device optical fiber cores, (ii) the number of device optical fiber gratings define reflected device optical signals based on a shape of the elongate medical device, (iii) the device optical fiber is optically coupled with the console, and (iv) the received device tracking signals include the reflected device optical signals propagated proximally along the device optical fiber.

In some embodiments, the landmarking device and the elongate medical device are simultaneously coupled with the console. In some embodiments, at least one of the landmarking device or the elongate medical device is optically coupled with the console, and at least the other one of the landmarking device or the elongate medical device is magnetically coupled with the console. In some embodiments, the landmarking device and the elongate medical device are simultaneously magnetically coupled with the console. In some embodiments, the landmarking device and the elongate medical device are simultaneously optically coupled with the console. In some embodiments, the landmarking device and the elongate medical device are simultaneously optically and magnetically coupled with the console.

In some embodiments, the landmarking device includes a catheter and, in some embodiments, the landmarking device is configured for insertion within a lumen of a vascular catheter.

Also disclosed herein is a method of inserting a medical into a patient that, according to some embodiments, includes receiving by a system module landmarking signals originating from a plurality of landmarking elements disposed along an elongate landmarking device of a medical system, where the landmarking device is disposed across an exterior of the patient. The landmarking elements are coupled with the patient and are arranged on the patient in accordance with a desired insertion pathway for the elongate medical device. The method further includes defining one or more digital landmarks of the landmarking device based on the received landmarking signals. The method further includes receiving by the system device tracking signals originating from a plurality of device tracking elements disposed along the elongate medical device and determining an actual pathway of the elongate medical device inserted into the patient, where the actual pathway is based the received device tracking signals originating from the device tracking elements.

In some embodiments, the method further includes storing the landmarking pathway in memory of the medical system, where the memory includes a non-transitory computer-readable medium.

In some embodiments, the method further includes depicting a landmarking pathway image in combination with an actual pathway image on a display of the system.

In some embodiments, the method further includes comparing the landmarking pathway with the actual pathway and providing an alert in response to the comparison when the actual pathway deviates from the landmarking pathway beyond a defined deviation limit.

In some embodiments of the method, the landmarking elements include landmarking magnetic elements, and the received landmarking signals include magnetic signals emanating from the landmarking magnetic elements.

In some embodiments of the method, the land marking device includes a shapable elongate member. In some embodiments, the landmarking magnetic elements are disposed along the shapable elongate member, and in some embodiments, the landmarking magnetic elements are positionable along the shapable elongate member. In such embodiments, the method may further include adjusting the position of one or more the number of landmarking magnetic elements along the shapable elongate member.

In some embodiments of the method, the landmarking device includes a landmarking optical fiber extending along the shapable elongate member, where the landmarking optical fiber includes multiple optical fiber cores having a number of optical fiber gratings disposed along the multiple optical fiber cores, and where the number of optical fiber gratings define reflected optical signals based on a shape of the shapable elongate member. In such embodiments, the landmarking optical fiber is optically coupled with the system module, and the received landmarking signals include the reflected optical signals propagated proximally along the landmarking optical fiber.

In some embodiments of the method, the device tracking elements include tracking magnetic elements disposed along the elongate medical device, and the received device tracking signals include magnetic signals emanating from the tracking magnetic elements.

In some embodiments of the method, the elongate medical device includes a device optical fiber extending along the elongate medical device, where the device optical fiber includes multiple optical fiber cores having a number of optical fiber gratings disposed along the multiple optical fiber cores, and where the number of optical fiber gratings define reflected device optical signals based on a shape of the elongate medical device. The device optical fiber is optically coupled with the system module, and the received device tracking signals include the reflected device optical signals propagated proximally along the optical fiber of the elongate medical device.

In some embodiments of the method, the landmarking device and the elongate medical device are simultaneously coupled with a console of the medical system. In some embodiments, at least one the landmarking device or the elongate medical device is optically coupled with the console, and at least the other one of landmarking device or the elongate medical device is magnetically coupled with the console. In some embodiments, the landmarking device and the elongate medical device are simultaneously magnetically coupled with the console. In some embodiments of the method, the landmarking device and the elongate medical device are simultaneously optically coupled with the console. In some embodiments of the method, the landmarking device and the elongate medical device are simultaneously optically and magnetically coupled with the console.

In some embodiments of the method, the landmarking device includes a catheter and in some embodiments of the method, the landmarking device is configured for insertion within a lumen of a vascular catheter.

Also disclosed herein is a landmarking device for mapping an insertion pathway of a vascular device that, according to some embodiments, includes a shapable elongate member configured for placement on a patient exterior along a desired internal pathway for the vascular device and a number of landmarking elements disposed along the shapable elongate member, where each landmarking element includes a characteristic detectable by the medical device tracking system.

In some embodiments of the device, the landmarking elements include landmarking magnetic elements configured to define a magnetic field detectable by a plurality of magnetometers of a medical device tracking system. In some embodiments, the landmarking magnetic elements are positionable along the shapable elongate member.

In some embodiments of the device, the landmarking elements include a landmarking optical fiber extending along the shapable elongate member, where the landmarking optical fiber includes multiple optical fiber cores having a number of optical fiber gratings disposed along the multiple optical fiber cores, and where the number of optical fiber gratings define reflected optical signals based on a shape of the landmarking optical fiber. The landmarking optical fiber is configured to optically couple with the medical device tracking system such that the reflected optical signals are detectable by the medical device tracking system.

In some embodiments of the device, the landmarking device includes a catheter, and in some embodiments, the landmarking device is configured for insertion with a lumen of a vascular catheter.

These and other features of the concepts provided herein will become more apparent to those of skill in the art in view of the accompanying drawings and following description, which disclose particular embodiments of such concepts in greater detail.

### DRAWINGS

Embodiments of the disclosure are illustrated by way of example and not by way of limitation in the figures of the accompanying drawings, in which like references indicate similar elements and in which:

FIG. 1 is an illustration of a medical system for inserting a medical device into a patient, in accordance with some embodiments;

FIG. 2 is a block diagram of a console of the system of FIG. 1, in accordance with some embodiments;

FIG. 3A is illustration of the system of FIG. 1 in use with a patient during a landmarking process, in accordance with some embodiments;

FIG. 3B is illustration of the system of FIG. 1 in use with a patient during a medical device tracking process, in accordance with some embodiments; and

FIG. 4 is a flow chart of an exemplary method of the system of FIG. 1, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a vascular device disclosed herein includes a portion of the vascular device intended to be near a clinician when the vascular device is used on a patient. Likewise, a “proximal length” of, for example, the vascular device includes a length of the vascular device intended to be near the clinician when the vascular device is used on the patient. A “proximal end” of, for example, the vascular device includes an end of the vascular device intended to be near the clinician when the vascular device is used on the patient. The proximal portion, the proximal end portion, or the proximal length of the vascular device can include the proximal end of the vascular device; however, the proximal portion, the proximal end portion, or the proximal length of the vascular device need not include the proximal end of the vascular device. That is, unless context suggests otherwise, the proximal portion, the proximal end portion, or the proximal length of the vascular device is not a terminal portion or terminal length of the vascular device.

With respect to “distal,” a “distal portion” or a “distal end portion” of, for example, a vascular device disclosed herein includes a portion of the vascular device intended to be near or in a patient when the vascular device is used on the patient. Likewise, a “distal length” of, for example, the vascular device includes a length of the vascular device intended to be near or in the patient when the vascular device is used on the patient. A “distal end” of, for example, the vascular device includes an end of the vascular device intended to be near or in the patient when the vascular device is used on the patient. The distal portion, the distal end portion, or the distal length of the vascular device can include the distal end of the vascular device; however, the distal portion, the distal end portion, or the distal length of the vascular device need not include the distal end of the vascular device. That is, unless context suggests otherwise, the distal portion, the distal end portion, or the distal length of the vascular device is not a terminal portion or terminal length of the vascular device.

The phrases “connected to,” “coupled with,” and “in communication with” refer to any form of interaction between two or more entities, including but not limited to mechanical, electrical, optical, magnetic, communicative, and operative interaction. Two components may be coupled with each other even though they are not in directly coupled with each other. For example, two components may be coupled with each other through an intermediate component.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified. Additional methods may comprise a subset of the one or more steps or actions.

References to approximations may be made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially straight” is recited with respect to a feature, it is understood that in further embodiments, the feature can have a precisely straight configuration.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

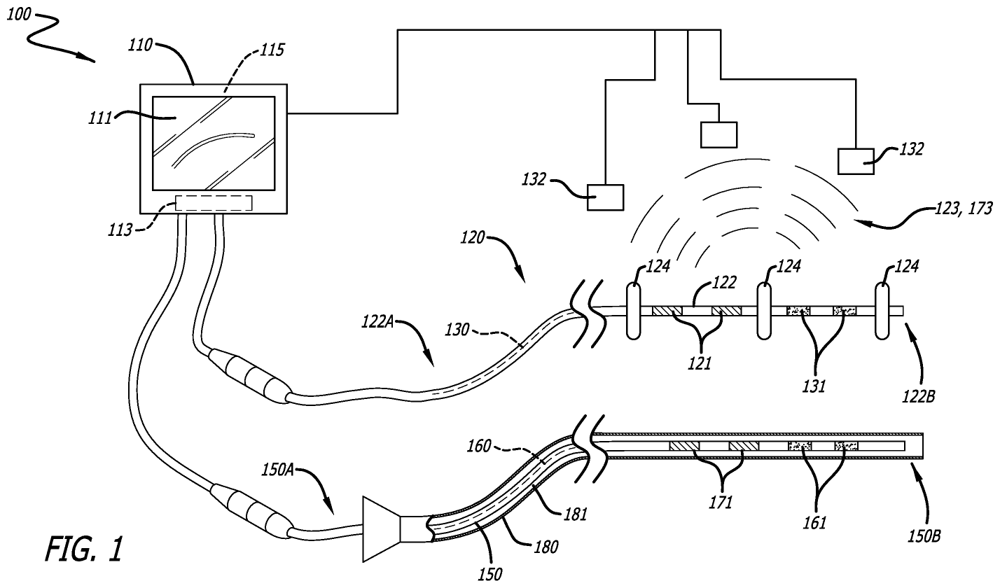

FIG. 1 is an illustration of a medical system for landmarking a vascular pathway for an elongate medical device, such as a catheter, for example. The medical system 100 is generally configured to define one or more digital landmarks in accordance with a desired insertion pathway for an elongate medical device. The system 100 may further be configured to determine/define a landmarking pathway based on the one or more digital landmarks. In some embodiments, the medical system (system) 100 may be further configured to track the location of the elongate medical device (i.e., determine the actual pathway of the elongate medical device) with respect the digital landmarks and/or the landmarking pathway. The system 100 generally includes a system module 110 and a landmarking device 120, where the landmarking device 120 is operatively coupled with the system module 110, the system module 110 including a console 115. The landmarking device 120 is configured to define the digital landmarks, where the digital landmarks are composed of data pertaining to characteristics of the landmarking device 120 as described further below. The characteristics are detectable by the system module 110 via landmarking signals defined by the landmarking elements of the landmarking device 120. The system module 110 may also include a user interface 113 enabling input from a clinician. In some embodiments, the user interface 113 may include one or more input devices, such as a touch screen, push buttons, or a microphone, for example.

In some embodiments, the landmarking device 120 includes a shapable elongate member 122 extending between a proximal end 122A and a distal end 122B. The elongate member 122 is configured for placement atop a patient, such as atop a front side of the patient, for example. The elongate member 122 is shapable similar to a catheter or a guidewire, for example, such that the elongate member 122 may be manipulated into a shape consistent with a vascular pathway within the patient. In some embodiments, the elongate member 122 may be plastically shapable such that the elongate member 122 maintains a desired shape after manual manipulation by the clinician. In some embodiments, the elongate member 122 may include a number (e.g., 1, 2, 3, 4 or more) adhesive members (or portions) 124 configured to attach the elongate member 122 to a skin of the patient, so that the position and the shape of the elongate member 122 remains fixed during use.

In the illustrated embodiment, the landmarking device 120 includes a landmarking optical fiber 130 extending along the length of the landmarking device 120, the landmarking optical fiber 130 operatively coupled with the system module 110. The landmarking optical fiber 130 is a multicore optical fiber including a plurality of fiber optic gratings 131 (e.g., fiber optic Bragg gratings) disposed along the length of the landmarking optical fiber 130, where the fiber optic gratings 131 are configured to enable shape sensing of the landmarking device 120 by logic of the system module 110. The multicore optical fiber includes multiple optical fiber cores where each optical fiber core includes a plurality of fiber optic Bragg gratings where each fiber optic Bragg is configured to define a reflected optical signal including a spectral width of broadband light based on a strain of the optical fiber core. The optical fiber cores are spaced a distance radially away from a central axis of the multicore optical fiber such a strain experienced optical fiber is related to the shape of the multicore optical fiber. As such, reflected optical signals of different spectral widths can be processed by logic of the system to determine the shape of the landmarking optical fiber 130.

In the illustrated embodiment, the landmarking device 120 is configured to enable tracking or mapping of the location of the landmarking device 120 during use, such as during as during and/or after placement of the landmarking device 120 on the patient. The landmarking device 120 includes a number (e.g., 2, 3, 4, or more) of landmarking elements that may include landmarking magnetic elements 121 disposed along the elongate member 122 of the landmarking device 120, where the landmarking magnetic elements 121 generate a magnetic field 123 detectable by a number of magnetometers 132 of the system 100. The landmarking magnetic elements 121 may be passive magnets or magnetized ferrous elements of the landmarking device 120. The landmarking magnetic elements 121 may include dipoles having a length, an orientation, and a spacing between adjacent dipoles. In some embodiments, the length and orientation of the dipoles combined with the spacing between adjacent dipoles may define a magnetic signature of the landmarking device 120, where the magnetic signature may be distinct from other magnetic signatures of other landmarking devices. The landmarking magnetic elements 121 define the magnetic field 123, e.g., a combination of multiple magnetic fields individually defined by each of the landmarking magnetic elements 121. The magnetic field 123 enables determining the position and shape of the elongate member 122 of the landmarking device 120 by the system module 110. According to some embodiments, the landmarking device 120 may include both the landmarking optical fiber 130 and the landmarking magnetic elements 121 or either of the landmarking optical fiber 130 or the landmarking magnetic elements 121. In some embodiments, the landmarking magnetic elements 121 may be separate individual magnets not coupled with each other.

In some embodiments, the system 100 further includes an elongate medical device (medical device) 150, operatively coupled with the system module 110. The medical device 150 is operatively coupled with the system module 110. The medical device 150 extends between a proximal end 150A and a distal end 150B. In the illustrated embodiment, the medical device 150 includes a device optical fiber 160 extending along the length of the medical device 150. The device optical fiber 160 is multicore optical fiber includes similar structure and functionality of the landmarking optical finer 130 as described above to define shape sensing capability of the medical device 150. As such, logic of the system module 110 may determine a shape of the medical device.

In the illustrated embodiment, the medical device 150 is configured to enable tracking or mapping of the location of the medical device 150 during use, such as during and/or after placement of the medical device 150 within the patient. The medical device 150 includes a number (e.g., 1, 2, 3, 4, or more) of device magnetic elements 171 disposed along the medical device 150. The device magnetic elements 171 may be passive magnets or magnetized ferrous elements of the medical device 150. The magnetic elements 131 may include dipoles having a length, an orientation, and a spacing between adjacent dipoles. In some embodiments, the length and orientation of the dipoles combined with the spacing between adjacent dipoles may define a magnetic signature of the medical device 150, where the magnetic signature may be distinct from other magnetic signatures of other medical devices. The device magnetic elements 171 define a magnetic field 173 (similar to the magnetic field 123), e.g., a combination of multiple magnetic fields individually defined by each of the device magnetic elements 171. The magnetic field 173 enables magnetic tracking of the medical device 150 by logic of the system module 110. According to some embodiments, the medical device 150 may include both the device optical fiber 160 and the device magnetic elements 171 or either of the device optical fiber 160 or the device magnetic elements 171.

In some embodiments, the medical device 150 may take the form of a guidewire, a stylet, a catheter, or any device suitable for insertion within the vasculature of the patient and/or insertion within a lumen 181 of a catheter 180. In some embodiments, the landmarking device 120 may be the medical device 150. As such, the clinician may place the medical device 150 (which may also include a catheter) atop the patient during a landmarking process. Similarly, the clinician may insert the landmarking device 120 into the lumen 181 of the catheter 180 during a medical device tracking process.

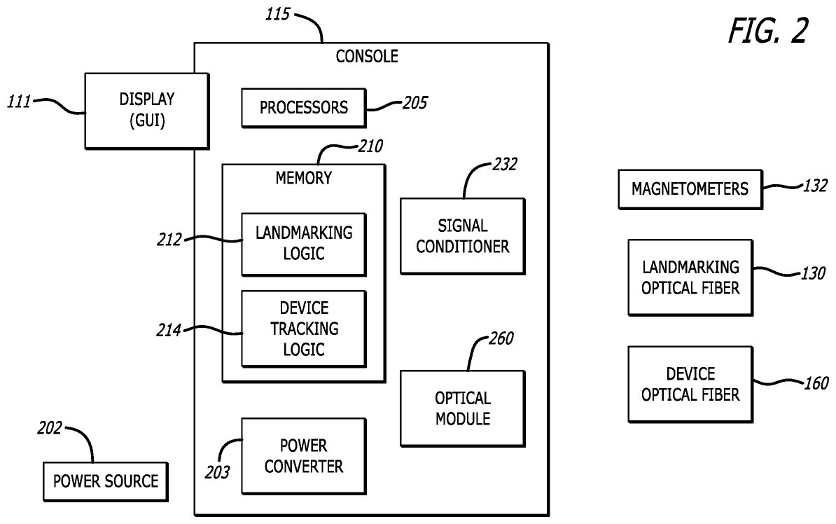

FIG. 2 illustrates a block diagram of the console 115, in accordance with some embodiments. The console 115 receives power from an external power source 202, and a power converter 203 defines and distributes electrical power to the other console components. The console 115 includes a number of processors 205 coupled with memory 210 including a non-transitory computer-readable medium. Logic stored in the memory 210 includes landmarking logic 212 and device tracking logic 214. The magnetometers 132 are couple with the console 115 and receive power from the console 115 via the power converter 203, and a signal conditioner 232 receives and converts magnetometer signals into magnetometer data for processing by the logic.

An optical module 260 is optically coupled with each of the landmarking optical fiber 130 and the device optical fiber 160. In some embodiments, both the landmarking optical fiber 130 and the device optical fiber 160 may be simultaneously coupled with the optical module 260. In other embodiments, each the landmarking optical fiber 130 and the device optical fiber 160 may be individually coupled with the optical module 260 at different times. In some embodiments, the optical module 260 may be configured to couple with only one optical fiber at a time. For example, the optical module 260 may be coupled with the landmarking optical fiber 130 during a first duration of use. Thereafter, the optical module 260 may be decoupled from the landmarking optical fiber 130, and optical module 260 may then be coupled with the device optical fiber 160. The signal conditioner 232 and the optical module 260 are coupled with the processors 205.

Generally speaking, the landmarking logic 212 processes magnetometer data associated with the magnetic field 123 and optical data from the optical module 260 associated with the landmarking optical fiber 130. Similarly, the device tracking logic 214 processes magnetometer data associated with the magnetic field 173 and optical data from the optical module 260 associated with the device optical fiber 160. Specific system operations that may be performed by the landmarking logic 212 and the device tracking logic 214 are described in further detail below.

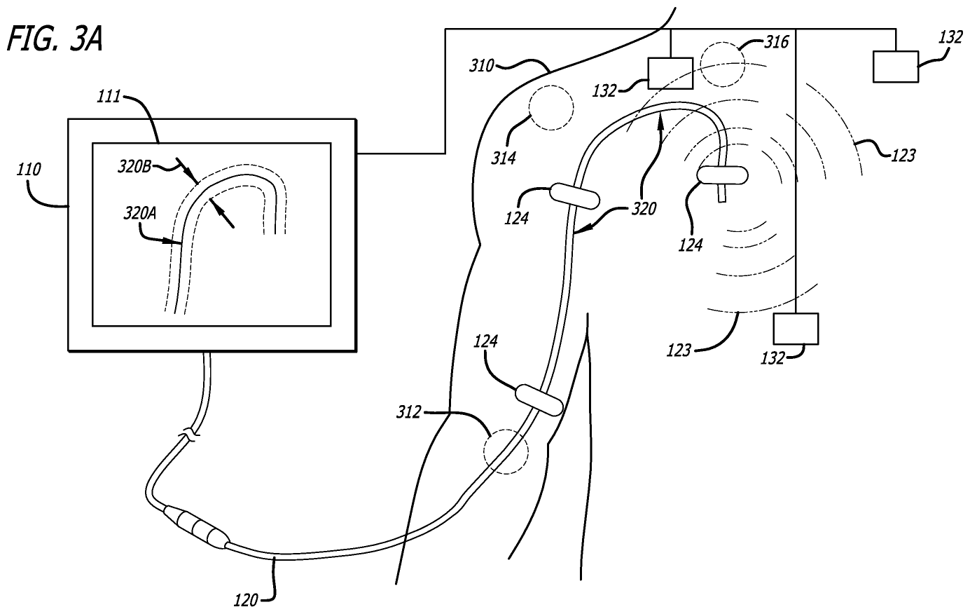

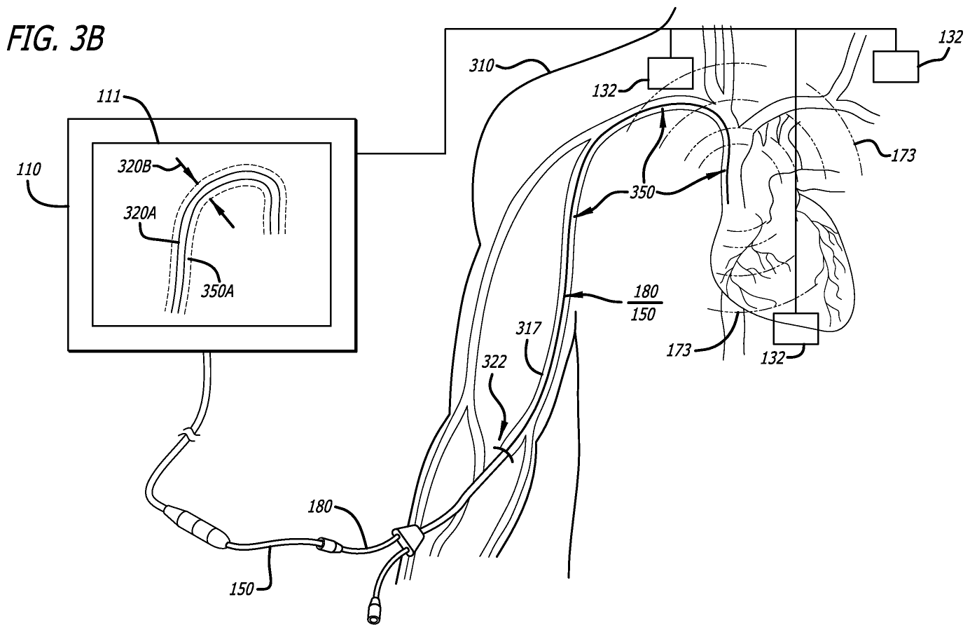

FIG. 3A illustrates the system 100 in use with a patient 310 during a landmarking process. The magnetometers 132 are placed on the patient 310 and coupled with the system module 110. The landmarking device 120 is coupled with the system module 110 and the elongate member 122 of the landmarking device 120 is placed atop the patient 310. The elongate member 122 is shaped and positioned to define a landmarking pathway 320 corresponding with a desired vascular pathway for the medical device 150 (not shown in FIG. 3A, see FIG. 1). The clinician has manipulated the position and shape of the elongate member 122 in accordance with a number of anatomical landmarks, such as an intended insertion site 312, a shoulder 314, and/or a clavicle head 316, for example, to define the landmarking pathway 320 including the digital landmarks. In some embodiments, the clinician may attach the landmarking device 120 to the skin of the patient 310 via any number of the adhesive members 124.

The landmarking logic 212 may determine the landmarking pathway 320, including the digital landmarks, from landmarking signals that include magnetometer data (e.g., magnetic signals) defined by the magnetic field 123. More specifically, the landmarking logic 212 may determine the location of the magnetic elements 121 (see FIG. 1) with respect to the magnetometers 132. As the magnetometers 132 are placed on the patient 310 at locations with respect the anatomical landmarks, the landmarking logic 212 may determine the positions of the magnetic elements 121 with respect to the anatomical landmarks. The landmarking logic 212 may define the digital landmarks based on the positions of the magnetic elements 121. As such, the landmarking logic 212 may determine the shape and position of the landmarking device 120 with respect to the anatomical landmarks, thereby determining or defining the landmarking pathway 320.

Having determined the landmarking pathway 320, the landmarking logic 212 may depict a landmarking pathway image 320A on the display 111. The landmarking pathway image 320A on the display 111 provides the clinician with a pathway reference that provides guidance to the clinician when inserting the medical device 150. In some embodiments, the landmarking logic 212 may depict the landmarking pathway image 320A on the display in response to a landmarking input. In some embodiments, the landmarking input may include input from the clinician, such pressing a button, or issue a voice command, for example. In some embodiments, the landmarking input may be related to determining the landmarking pathway 320. For example, the landmarking logic 212 may compare a shape of the landmarking device 120 with a landmarking device shape stored in memory. In response to the comparison, the landmarking logic 212 may initiate depicting the landmarking pathway image 320A when the landmarking pathway 320 matches the landmarking device shape stored in memory in accordance with predefined matching criteria.

The landmarking logic 212 may also store the landmarking pathway 320 in the memory 205. The landmarking logic 212 may store the landmarking pathway 320 in response to receiving input from the clinician.

The landmarking logic 212 may also determine the landmarking pathway 320 (i.e., a shape of the landmarking pathway 320) via landmarking signals that include optical data acquired from the landmarking optical fiber 130. More specifically, the landmarking logic 212 may include shape sensing logic. The shape sensing logic may receive optical data from the optical module 260, where the optical data are based on optical signals reflected off of the fiber optic gratings 131 of the optical fiber 130 and further where the reflected optical signals relate to a strain of the optical fiber 130 at each of the fiber optic gratings 131. As such, the landmarking logic 212 may process the optical data to determine the shape of the landmarking device 120, where the shape of the landmarking device 120 may at least partially define the landmarking pathway 320.

In some embodiments, the landmarking logic 212 may determine the landmarking pathway 320 only via the magnetic elements 121. In other embodiments, the landmarking logic 212 may determine the landmarking pathway 320 only via the optical fiber 130. In further embodiments, the landmarking logic 212 may determine the landmarking pathway 320 via the magnetic elements 121 in combination with the optical fiber 130.

Having determined the landmarking pathway 320, the landmarking logic 212 may define a deviation limit 320B disposed about the landmarking pathway 320. The deviation limit 320B may be defined in accordance with anatomical structure, such as an inside diameter of a blood vessel, for example.

FIG. 3B illustrates the system 100 in use with a patient 310 during the medical device tracking process. The magnetometers 132 are placed on the patient 310 in the same location as during the landmarking process illustrated in FIG. 3A and coupled with the system module 110. The landmarking device 120 is removed from the patient 310. The medical device 150 is inserted through the insertion site 322 and disposed within desired vascular pathway 317. The medical device 150 is also coupled with the system module 110. With the medical device disposed within the desired vascular pathway 317, the medical device 150 is shaped and positioned by the vascular pathway 317 to define an actual pathway 350.

The device tracking logic 214 may determine the actual pathway 350 via the magnetometer data defined by the magnetic field 173. More specifically, the device tracking logic 214 may determine the location of the magnetic elements 171 (see FIG. 1) with respect to the magnetometers 132. The device tracking logic 214 may determine the positions of the magnetic elements 171 with respect to the magnetometers 132. As the magnetometers 132 are placed at the same locations as when the landmarking pathway 320 was determined, the device tracking logic 214 may determine the shape and position of the medical device 350 with respect to the landmarking pathway 320. Having determined the actual pathway 350, the device tracking logic 214 may depict an actual pathway image 350A on the display 111 along with the landmarking pathway image 320A. As such, the clinician my observe the actual pathway image 350A in relation to the landmarking pathway image 320A during and after insertion of the medical device 150.

The device tracking logic 214 may also determine the actual pathway 350 (i.e., a shape of the actual pathway 350) via optical data acquired from the device optical fiber 160. Similar to the landmarking logic 212, the device tracking logic 214 may include shape sensing logic, and thereby in the same fashion, determine a shape of the device optical fiber 160. As such, the device tracking logic 214 may determine the shape of the medical device 150 which defines the actual pathway 350.

In some embodiments, the device tracking logic 214 may determine the actual pathway 350 only via the magnetic elements 171. In other embodiments, the device tracking logic 214 may determine the actual pathway 350 only via the optical fiber 160. In further embodiments, the device tracking logic 214 determine the actual pathway 350 via the magnetic elements 171 in combination with the optical fiber 160.

The device tracking logic 214 may compare the actual pathway 350 with the landmarking pathway 320. The comparison may also include evaluating the actual pathway 350 in relation to the deviation limit 320B to determine if the distal end 150B of the medical device 150 remains within the deviation limit 320B during insertion. In an instance where the distal end 150B or any portion of the actual pathway 350 exceeds the deviation limit 320B, the device tracking logic 214 may generate an alert to notify the clinician that medical device 150 may not be disposed within the desired vascular pathway 317.

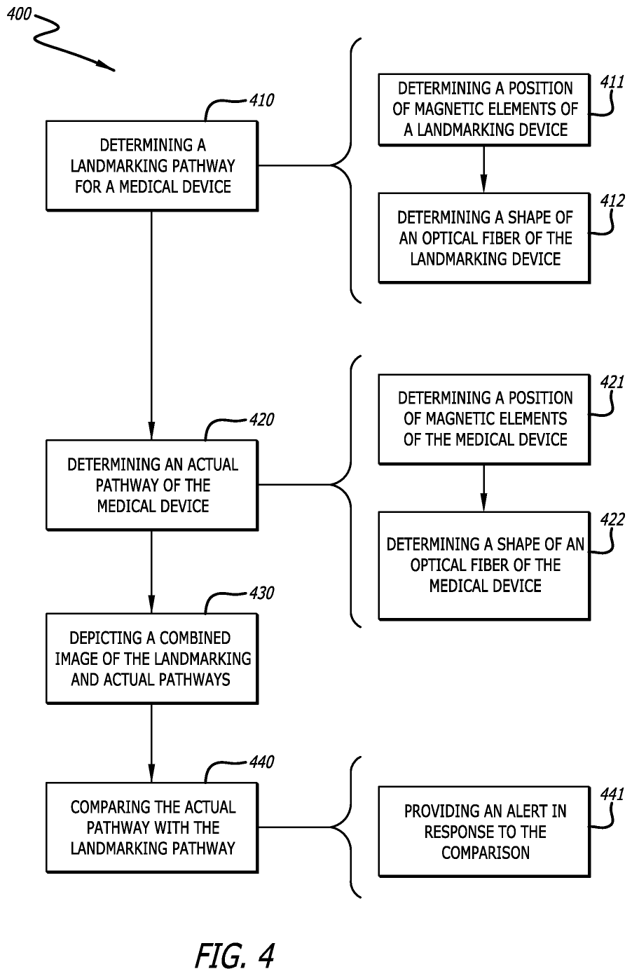

FIG. 4 illustrates a flow chart of an exemplary method of inserting a medical device into a patient that includes all or a subset of the following steps or processes as performed by the system as executed by processers according to logic modules of the system. The method 400 includes determining a landmarking pathway for the medical device (block 410), where determining a landmarking pathway includes receiving by the system module a number of landmarking signals originating from a number of landmarking elements of a landmarking device. In some embodiments, the landmarking elements are attached to a shapable elongate member of the landmarking device, where the landmarking device is arranged (shaped and positioned) to define a landmarking pathway related to a desired internal pathway for the elongate medical device.

In some embodiments, determining a landmarking pathway includes determining the position of a number of the landmarking magnetic elements placed on an exterior the patient (block 411). The landmarking magnetic elements may be coupled with each other via the elongate member or the magnetic elements may be separate magnetic elements (e.g., magnets) placed individually on the patient. In some embodiments, the landmarking magnetic elements are positionable along the shapable elongate member.

In some embodiments, determining a landmarking pathway includes determining a shape of the optical fiber of the landmarking device (block 412). In some embodiments, the landmarking device includes a landmarking optical fiber extending along the shapable elongate member, where the landmarking optical fiber includes multiple optical fiber cores having a number of optical fiber gratings disposed along the multiple optical fiber cores, and where the number of optical fiber gratings define reflected optical signals based on a shape of the shapable elongate member. In such embodiments, the landmarking optical fiber is optically coupled with the console, and the received landmarking signals include optical signals propagated proximally along the optical fiber of the landmarking device.

In some embodiments, determining a landmarking pathway may include depicting an image of the landmarking pathway on the display so that the clinician may observe the landmarking pathway and make any needed adjustments to the shape and/or position of the landmarking device.

In some embodiments, determining a landmarking pathway may include storing the landmarking pathway in memory of the medical system. Storing the landmarking pathway in memory may allow the clinician to remove the landmarking device from the patient. In some embodiments, storing the landmarking pathway may include receiving input from the clinician, such as a touch input or voice input, for example.

The method 400 further includes determining an actual pathway of the elongate medical device (i.e., tracking the location of the medical device) during and/or after insertion into the patient (block 420), where determining the actual pathway includes receiving a number of device tracking signals originating from a number of device tracking elements of medical device. In some embodiments, determining an actual pathway may include determining the position of the tracking magnetic elements disposed along the elongate medical device (block 421).

In some embodiments, determining a landmarking pathway includes determining a shape of the optical fiber of the medical device (block 422), where the device optical fiber includes multiple optical fiber cores having a number of optical fiber gratings disposed along the multiple optical fiber cores, and where the number of optical fiber gratings define reflected optical signals based on a shape of the medical device.

The method 400 further includes depicting the actual pathway on a display of the system in combination with the landmarking pathway (block 430) so that the clinician may observe the position and/or shape of the actual pathway in relation to the landmarking pathway.

The method 400 further includes comparing the actual pathway with the landmarking pathway (block 440) to determine if the actual pathway deviates from the landmarking pathway. In some embodiments, comparing the actual pathway with the landmarking pathway includes providing an alert in response to the comparison (block 441) when the actual pathway deviates from the landmarking pathway beyond a defined deviation limit.

In some embodiments of the method 400, the landmarking device and the elongate medical device are simultaneously coupled with the console of the medical system. In some embodiments of the method 400, at least one the landmarking device or the elongate medical device is optically coupled with the console, and at least the other one of landmarking device or the elongate medical device is magnetically coupled with the console. In some embodiments of the method 400, the landmarking device and the elongate medical device are simultaneously magnetically coupled with the console. In some embodiments of the method 400, the landmarking device and the elongate medical device are simultaneously optically coupled with the console. In some embodiments of the method 400, the landmarking device and the elongate medical device are simultaneously optically and magnetically coupled with the console.

In some embodiments of the method 400, the landmarking device includes a catheter, e.g., the magnetic elements and/or the optical fiber may be integral to the catheter. In other embodiments of the method 400, the landmarking device is configured for insertion within a lumen of a vascular catheter, i.e., the landmarking device may be a guidewire, a stylet, or any other suitable device that may be inserted within the lumen of the catheter.

A method of use for the system 100 may include all or a subset of the flowing steps or processes as performed by the clinician. The method of use may include coupling the landmarking device with the system module. The method of use may include placing the magnetometers on the patient. The method of use may include placing the landmarking device on the patient in accordance with the defined vascular pathway for the medical device. In some embodiments, placing the landmarking device on the patient includes placing individual magnets on the patient at defined landmark locations. In some embodiments, placing the landmarking device on the patient includes attaching one or more adhesive members to the patient to secure the landmarking device to the patient at the desired position and having the desired shape. The method of use may include providing input to the system to cause the system 100 to depict the landmarking pathway image on the display. The method of use may include providing input to the system to cause the system to store landmarking pathway in memory. The method of use may include removing the landmarking device from the patient. The method of use may include coupling the medical device with the system module and inserting the medical device into the patient. The method of use may include providing input to the system to cause the system to depict the actual pathway image on the display together with landmarking image. The method of use may include observing a live actual pathway image in relation the landmarking pathway image on the display during insertion of the medical device.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A medical system, comprising:
an elongate landmarking device configured for placement across an exterior of the patient, the landmarking device, comprising a plurality of landmarking elements extending along a length of the landmarking device; and
a system module comprising a console operably coupled with the landmarking device, the console having a number of processors and memory including a non-transitory computer-readable medium, having logic stored thereon that, when executed by the processors, performs operations, including:
receiving, by the system module, landmarking signals originating from the plurality of landmarking elements; and
defining one or more digital landmarks of the landmarking device based on the received landmarking signals, wherein:
the plurality of landmarking elements are arranged along the landmarking device to define the one or more digital landmarks, and
the digital landmarks are configured to indicate a desired insertion pathway for an elongate medical device.

2. The system according to claim 1, wherein the operations further include defining a landmarking pathway based on the one or more digital landmarks.

3. The system according to claim 2, wherein the operations further include storing the landmarking pathway in memory.

4. The system according to claim 1, wherein:
the landmarking elements include landmarking magnetic elements, and
the received landmarking signals include magnetic signals emanating from the landmarking magnetic elements.

5. The system according to claim 1, wherein the landmarking device includes a shapable elongate member.

6. The system according to claim 5, wherein the landmarking magnetic elements are disposed along the shapable elongate member.

7. The system according to claim 6, wherein the landmarking magnetic elements are positionable along the shapable elongate member.

8. The system according to claim 5, wherein:
the landmarking elements include a landmarking optical fiber extending along the shapable elongate member,
the landmarking optical fiber includes multiple optical fiber cores having a number of optical fiber gratings disposed along the multiple optical fiber cores,
the number of optical fiber gratings define reflected optical signals based on a shape of the shapable elongate member,
the landmarking optical fiber is optically coupled with the console, and
the received landmarking signals include the reflected optical signals propagated proximally along the landmarking optical fiber.

9. The system according to claim 1, further comprising:
an elongate medical device configured for insertion within a patient, wherein the elongate medical device:
is operably coupled with the console, and
includes a number of device tracking elements disposed along the elongate medical device.

10. The system according to claim 9, wherein the operations further include:
receiving, by the system module, device tracking signals originating from the device tracking elements disposed along the elongate medical device; and
determining an actual pathway of the elongate medical device inserted into the patient, the actual pathway based on the received device tracking signals.

11. The system according to claim 10, wherein:
the console is coupled with a display, and
the operations further include depicting an image of the landmarking pathway in combination with an image of the actual pathway on the display.

12. The system according to claim 10, wherein the operations further include:
comparing the landmarking pathway with the actual pathway; and
providing an alert in response to the comparison when the actual pathway deviates from the landmarking pathway beyond a defined deviation limit.

13. The system according to claim 9, wherein:
the device tracking elements include tracking magnetic elements disposed along the elongate medical device, and
the received device tracking signals include magnetic signals emanating from the tracking magnetic elements.

14. The system according to claim 13, wherein the landmarking device and the elongate medical device are simultaneously coupled with the console.

15. The system according to claim 13, wherein:
at least one of the landmarking device or the elongate medical device is optically coupled with the console, and
at least the other one of the landmarking device or the elongate medical device is magnetically coupled with the console.

16. The system according to claim 13, wherein the landmarking device and the elongate medical device are simultaneously magnetically coupled with the console.

17. The system according to claim 9, wherein:
the elongate medical device includes a device optical fiber extending along the elongate medical device,
the device optical fiber includes multiple device optical fiber cores having a number of device optical fiber gratings disposed along the multiple device optical fiber cores,
the number of device optical fiber gratings define reflected device optical signals based on a shape of the elongate medical device,
the device optical fiber is optically coupled with the console, and
the received device tracking signals include the reflected device optical signals propagated proximally along the device optical fiber.

18. The system according to claim 17, wherein the landmarking device and the elongate medical device are simultaneously optically coupled with the console.

19. The system according to claim 17, wherein the landmarking device and the elongate medical device are simultaneously optically and magnetically coupled with the console.

20. The system according to claim 1, wherein the landmarking device includes a catheter.

21. The system according to claim 1, wherein the landmarking device is configured for insertion into a lumen of a vascular catheter.

22-40. (canceled)

41. A landmarking device for mapping an insertion pathway of a vascular device, comprising:
a shapable elongate member configured for placement on a patient exterior along a desired internal pathway for the vascular device; and
a number of landmarking elements disposed along the shapable elongate member, each landmarking element including a characteristic detectable by a medical device tracking system.

42. The device according to claim 41, wherein the landmarking elements include landmarking magnetic elements configured to define a magnetic field detectable by a plurality of magnetometers of the medical device tracking system.

43. The device according to claim 42, wherein the landmarking magnetic elements are positionable along the shapable elongate member.

44. The device according to claim 41, wherein:
the landmarking elements include a landmarking optical fiber extending along the shapable elongate member,
the landmarking optical fiber includes multiple optical fiber cores having a number of optical fiber gratings disposed along the multiple optical fiber cores, and
the number of optical fiber gratings define reflected optical signals based on a shape of the landmarking optical fiber,
the landmarking optical fiber is configured to optically couple with the medical device tracking system such that the reflected optical signals are detectable by the medical device tracking system.

45. The device according to claim 41, wherein the landmarking device includes a catheter.

46. The device according to claim 41, wherein the landmarking device is configured for insertion within a lumen of a vascular catheter.
